SEQUENCE LISTING (1 sequence)

SEQ ID NO: 1 - DNA, 18 bp, synthetic construct
  misc_feature 1..18: note = synthetic polynucleotide
  misc_feature 1..18: note = deoxythymine
  source 1..18: mol_type = other DNA
tttttttttttttttttt

(19) United States
(12) Patent Application Publication
(10) Pub. No.: US 2023/0193239 A1
(43) Pub. Date: Jun. 22, 2023

(54) POLYNUCLEOTIDE PURIFICATION WITH MONOLITH COLUMNS

(71) Applicant: Alexion Pharmaceuticals, Inc., New Haven, CT (US)

(72) Inventors: Jared DAVIS, Hamden, CT (US); Joanna DEBEAR, Cheshire, CT (US); Christopher CHENG, Reading, MA (US)

(21) Appl. No.: 18/057,489

(22) Filed: Nov. 21, 2022

Related U.S. Application Data
(63) Continuation of application No. 16/303,207, filed on Nov. 20, 2018, now abandoned.
(60) Provisional application No. 62/341,140, filed on May 25, 2016.

(51) Int. Cl. C12N 15/10; C12Q 1/6806

(52) U.S. Cl. CPC C12N 15/1006; C12Q 1/6806; C12Q 2523/31; C12Q 2565/137

(57) ABSTRACT

Described herein are methods of purifying polynucleotides, e.g., imRNA and oligonucleotides, e.g., probes, primers and siRNA, using monolithic columns with immobilized ligands coupled to the monolithic column. Also described are monolithic columns for purifying polynucleotides from a sample; and methods of preparing such columns.

### CROSS-REFERENCE TO RELATED APPLICATIONS

This application is a continuation of U.S. application Ser. No. 16/303,207, filed Nov. 20, 2018, which is a U.S. National Stage entry filed under 35 U.S.C. § 371 of International Application No. PCT/2017/34193, filed May 24, 2017, which claims the benefit of and priority to U.S. Provisional Application No. 62/341,140, filed May 25, 2016, the contents of each of which are incorporated herein by reference in their entirety.

### INCORPORATION BY REFERENCE OF SEQUENCE LISTING PROVIDED AS A COMPUTER READABLE FILE

The contents of the text file named “049386-541C01 US_Sequence Listing.xml” which was created on Nov. 30, 2022 and is 2,803 bytes in size, is hereby incorporated by reference in their entirety.

### BACKGROUND

Messenger RNA, or mRNA, is a key intermediary in the conversion of genetic information into biologically active proteins. Many aspects of biomedical research and drug development depend on the ability to obtain high-quality, purified mRNA. Several properties of mRNA, however, make its purification challenging. Relative to total RNA, mRNA exists in very low copy numbers in cells. Furthermore, mRNA is highly sensitive to degradation by RNase enzymes, further compounding the difficulties of purification.

Current methods of isolating mRNA take advantage of the poly-adenine (poly-A) tail present on mRNA molecules. Oligonucleotides consisting of stretches of the nucleic acid base, deoxythymine (oligo-dT), are used to bind to the complementary poly-A tails of mRNA molecules. Oligo-dT-cellulose affinity chromatography has been used to purify mRNA from total RNA fractions.

Purification of mRNA using traditional chromatographic methods, however, is inefficient. Cellulose and other particle-based chromatography columns contain small pore sizes causing slow diffusion of large biomolecules such as mRNA and large contaminants or other particles. Consequently, mRNA purification over traditional particle-based columns is characterized by low flow rates, poor yields and extensive processing time. This problem is further exacerbated when mRNA, formulated for therapeutic delivery, are to be purified, as such formulated mRNA are even larger than naked mRNA.

There remains a need for improved methods of purifying mRNA molecules and other polynucleotides, both naked and formulated, to high purity with a high yield and lower processing time.

### SUMMARY

Described herein are compositions and methods for purifying polynucleotides, both naked and formulated, e.g., mRNA formulated in lipid nanoparticles (LNPs). Polynucleotides are purified from contaminants, such as, for example, other biomolecules, such as DNA, ribosomal and transfer RNA, and proteins, using monolithic column chromatography. Where formulated polynucleotides are purified, contaminants also include unformulated polynucleotide (“free” or “naked” polynucleotides).

In one embodiment, the disclosure is directed to a method of separating a formulated polynucleotide from free polynucleotide, the method comprising: a) loading a sample onto a monolith matrix comprising a ligand comprising: i) a reactive moiety coupled to the monolith matrix, and ii) an affinity moiety that binds to the free polynucleotide but not the formulated polynucleotide, wherein the ligand is immobilized to the monolithic matrix via the reactive moiety; and b) collecting the formulated polynucleotide from the column while the free polynucleotide remains immobilized on the monolith matrix. In a particular embodiment, the monolith matrix is contained in a column. In a particular embodiment, the formulated polynucleotide is a formulated mRNA. In a particular embodiment, the mRNA is formulated in a lipid nanoparticle. In a particular embodiment, the ligand is an oligo-dT probe. In a particular embodiment, the ligand is NH₂—C_{X}-dT_{Y}, where X is a whole number between 1 and 50 and Y is a whole number between 5 and 30. In a particular embodiment, the ligand is NH₂—C₁₂-dT₁₈. In a particular embodiment, the ligand further comprises a carbon linker positioned between the reactive moiety and the ligand. In a particular embodiment, the carbon linker is C_{X}, where X is a whole number between about 1 and about 50. In a particular embodiment, the methods described herein further comprise eluting the free polynucleotide from the monolith matrix by reducing the ionic strength of the liquid phase.

In one embodiment, the disclosure is directed to a method of purifying a polynucleotide from a sample, the method comprising: a) loading the sample onto a monolithic matrix comprising a ligand comprising: i) a reactive moiety coupled to the monolithic matrix, and ii) a ligand that binds to the polynucleotide, wherein the ligand is immobilized to the monolithic matrix via the reactive moiety; b) allowing for the polynucleotide to bind to the ligand; and c) eluting the polynucleotide from the monolith matrix after one or more contaminants have been substantially separated from the bound polynucleotide. In a particular embodiment, the reactive moiety is a primary amine. In a particular embodiment, the monolithic matrix is activated with an activating agent selected from carbonyldiimidazole, epoxy, ethylenediamine (EDA), carbodiimide, aldehyde, anhydride, imidoester and NHS ester. In a particular embodiment, the ligand further comprises a carbon linker positioned between the reactive moiety and the ligand. In a particular embodiment, the carbon linker is C_{X}, where X is a whole number between about 1 and about 50. In a particular embodiment, the polynucleotide is mRNA. In a particular embodiment, the ligand is an oligo-dT probe. In a particular embodiment, the ligand is NH₂—C_{X}-dT_{Y}, where X is a whole number between 1 and 50 and Y is a whole number between 5 and 30. In a particular embodiment, the ligand is NH₂—C₁₂-dT₁₈. In a particular embodiment, the methods described herein further comprise washing the column prior to eluting the polynucleotide, e.g., wherein the wash buffer contains a salt concentration of at least 200 mM, and the elution buffer contains a salt concentration of 100 mM or less, wherein the wash buffer comprises one or more salts selected from sodium sulfate, sodium chloride and sodium phosphate. In a particular embodiment, the elution buffer is selected from water and Tris. In a particular embodiment, the flow rate of the column is at least 0.5 mL/min or 0.5 CV/min (e.g., in a 1 mL column). Column volume is abbreviated “CV.”

In one embodiment, the disclosure is directed to a column for purifying a polynucleotide from a sample, said column comprising: a) a monolithic matrix; and b) a ligand comprising a reactive moiety coupled to the monolithic matrix, and a ligand that binds to the polynucleotide, wherein the ligand is immobilized to the monolithic matrix via the reactive moiety. In a particular embodiment, the reactive moiety is a primary amine. In a particular embodiment, the monolithic matrix is activated with an activating agent selected from carbonyldiimidazole, epoxy, ethylenediamine (EDA), carbodiimide, aldehyde, anhydride, imidoester and NHS ester. In a particular embodiment, the ligand is an oligo-dT probe. In a particular embodiment, the ligand further comprises a carbon linker positioned between the reactive moiety and the oligo-dT probe. In a particular embodiment, the carbon linker is C_{X}, where X is a whole number between 1 and 50. In a particular embodiment, the ligand is NH₂—C_{X}-dT_{Y}, where X is a whole number between 1 and 50 and Y is a whole number between 5 and 30. In a particular embodiment, the ligand is NH₂—C₁₂-dT₁₈.

In one embodiment, the disclosure is directed to a method of preparing a column described herein by a method comprising: a) treating the monolithic matrix with an activating agent to produce an activated monolithic matrix; and b) incubating the activated monolithic matrix in the presence of a ligand comprising a reactive moiety. In a particular embodiment, the activating agent is selected from carbonyldiimidazole, epoxy, ethylenediamine (EDA), carbodiimide, aldehyde, anhydride, imidoester and NHS ester.

### BRIEF DESCRIPTION OF THE DRAWINGS

FIG. 1 shows chromatogram traces from an RNA001-10-9 chromatography run at 200 mM sodium sulfate, 50 mM sodium phosphate, 1 0 mM EDTA pH 7 over the NH₂—C₁₂-dT₁₈ immobilized monolith disk column, as described in Example 2.

FIG. 2 shows a chromatogram (top) and glyoxyl gel image (bottom) from the RNA001 chromatography run described in Example 2. M=markers, S=starting material, D=linearized DNA template, C6-C9=main peak fractions.

FIG. 3 shows a chromatogram of RNA021 transcription (without poly-A tail) over the NH₂—C₁₂-dT₁₈-immobilized monolith disk, as described in Example 2.

FIG. 4 shows an overlay of chromatogram traces from RNA001 (with poly-A tail) and RNA021 (without poly-A tail) run under the same conditions on the NH₂—C₁₂-dT₁₈-immobilized monolithic column, as described in Example 2.

FIG. 5 shows a chromatogram of the transcription reaction of RNA001 tested on the NH₂—C₁₂-dT₁₈ monolith disk, as described in Example 3.

FIG. 6 shows RNA transcription reactions purified on an NH₂—C₁₂-dT₁₈ immobilized monolithic column and then run on a 1% E-GEL™ (precast agarose gel), as described in Example 3. The transcription reactions were loaded onto the monolithic column without further purification following transcription.

FIG. 7. shows an overlay of RNA001 chromatograms tested at 2, 3 and 4 mL/minute flow rate, as described in Example 4.

FIG. 8 is a map of DNA001 with two additional linearization sites to remove the poly-A tail, as described in Example 5.

FIG. 9 shows a chromatogram of RNA001 samples with and without the poly-A tail, as described in Example 5.

FIG. 10 shows a 1% E-GEL™ (precast agarose gel) of fractions eluted from an oligo-dT ligand immobilized monolithic column. The sample applied to the column contained RNA transcripts with and without poly-A tails, as described in Example 5.

FIG. 11 shows an overlay of chromatogram traces from RNA001 purified over two monolithic columns with oligo-dT₁₈ ligands (SEQ ID NO: 1) containing either a 6-carbon linker (C₆) or a 12-carbon linker (C₁₂), as described in Example 6. The trace labeled * is the A₂₆₀ of RNA001 purified over the C₁₂ oligo-dT monolith, and the trace labeled ** is the A₂₆₀ of RNA001 purified over the C₆ oligo-dT monolith.

FIG. 12 is an overlay of chromatograms of RNA001 purified using sodium sulfate- vs. sodium chloride-based buffers (overlapping traces are labeled with *).

FIG. 13 shows a chromatogram of RNA001 binding to the 1 mL NH₂—C₁₂-dT₁₈ monolith and elution using 10 mM Tris pH 7.5.

FIG. 14 is a set of gels showing separation of free mRNA, which is eluted (lane 10, top panel) and LNP-formulated mRNA (which comes off in the flow-through, lanes 1-6, bottom panel). The top panel shows intact, LNP-formulated mRNAs loaded onto a gel; the bottom panel shows the same LNP-formulated mRNAs after the LNP has been treated with detergent to lyse the LNP (note the “smiling” of the gel was due to high salt concentrations of the samples during lysis). For both panels, Lane M=Sample Load, Lanes 1-6=flow-through, Lanes 7-8=wash and Lane 10=elution. Note mRNA in wells on the top gel, mRNA within LNPs have hindered electrophoretic mobility. Lysing of the LNPs shows that the load and flow-through fractions contained mRNA that was sequestered in LNPs.

FIG. 15 is a bar graph showing the improvement of encapsulation efficiency (EE) that results from LNP purification from free mRNA. LNP load materials with EE=84-87% were purified to EE=94-96%. Spiking LNPs with free mRNA (1:1) was purified from EE=53% to EE=79%. The yield for the purification procedure was −70%.

FIGS. 16A-C are chromatograms (A260) showing elution profiles for various buffer gradients that were tested for purifying mRNA (RNA025). The top line represents the gradient (% buffer B), and the bottom line is the chromatogram. Oligonucleotides complementary to the 5′ end of the mRNA were designed following the T7 polymerase start site (18mer and 24mer oligos were tested; 18mer results are shown). For each oligonucleotide, a 6-carbon modifier was attached to the 3′ end and then conjugated to the monolith. FIG. 16A shows the results of a gradient wash. FIG. 16B shows results using a step wash at a conductivity level just before material starts to elute in the gradient wash (FIG. 16A). This step wash resulted in a significantly sharper elution peak in 10 mM Tris. A significant amount of material was still bound to the column, however, and only removed by a NaOH cleaning step for both of the chromatography runs. Therefore, a step elution at 2M, 4M, 6M and 8M urea was tested (FIG. 16C). As the A260 trace of the chromatogram shows, the RNA bound to the column was completely removed during the urea step elution before reaching the 10 mM NaOH cleaning step. Going forward, 4M urea was selected as the elution condition for all subsequent chromatography purification testing as the majority of the RNA was eluted from the monolith under these conditions.ALK2=5′ oligo(18)C6dT 3′. Buffer B: 50 mM sodium phosphate, 10 mM EDTA, pH=7.0.

### DETAILED DESCRIPTION

Described herein are compositions and methods of purifying polynucleotides and formulated polynucleotides, e.g., DNA, or RNA, e.g., mRNA, oligonucleotides, e.g., probes, primers and siRNA, or artificial or synthetic polynucleotides, from contaminants. Contaminants include, for example, other biomolecules, such as DNA, ribosomal and transfer RNA and proteins. In the case of formulated nucleotides, e.g., polynucleotides enveloped within a lipid nanoparticle (LNP), contaminants also included unformulated nucleotides (“free” polynucleotides). The materials and methods described herein comprise using monolithic column chromatography. The materials and methods described herein relate to unexpected findings that immobilization of polynucleotide ligands, e.g., oligo-deoxythymine (oligo-dT) and sequence-specific or non-specific oligonucleotides or affinity moieties, on monolithic chromatography columns allows for improved purification of polynucleotides, e.g., polynucleotides comprising poly-A. As described herein, any affinity moiety, e.g., a sequence-specific polynucleotide, can be used in conjunction with monolith columns to achieve polynucleotide purification, e.g., separation of formulated polynucleotides from free polynucleotides. The methods described herein are applicable to immobilizing a ligand via an active moiety to an activated monolith matrix, wherein the ligand specifically binds to the polynucleotide to be purified, e.g., through sequence-specific binding, through hybridization or other base-pairing interactions, or through chemical and non-chemical interactions.

The present disclosure is not limited to the particular embodiments of the disclosure described below, as variations of the particular embodiments can be made that still fall within the scope of the appended Claims. The terminology employed is for the purpose of describing particular embodiments, and is not intended to be limiting. The singular forms “a,” “an” and “the” include plural reference unless the context clearly dictates otherwise. Unless defined otherwise, all technical and scientific terms used herein have the same meaning as commonly understood to one of ordinary skill in the art.

Described herein is a solid support medium comprising attached polynucleotides or affinity ligands for the purification of biomolecules that specifically bind to the attached polynucleotides or affinity ligands. The solid support medium, for example, can be a column used to purify mRNA from a sample, said column comprising a monolithic matrix coupled to, for example, a ligand comprising an oligo-dT probe. The material of interest to be purified, for example, can be the material that binds to the ligand. Alternatively, the material of interest to be purified can be material that does not bind to the ligand, with a primary contaminant being bound to the ligands instead.

The terms “monolith,” “monolithic matrix” and “monolithic column” are used interchangeably herein to refer to a chromatography column composed of a continuous stationary phase made of a polymer matrix. In contrast to particle-based chromatography columns, monolithic columns are made of a porous polymer material with highly interconnected channels and large pore size. While particle-based columns rely on diffusion through pores, separation by monolithic columns occurs primarily by convective flow through relatively large channels (about 1 micron or more). Monolithic columns are commercially available and have been used to purify large biomolecules such as viruses, plasmid DNA, and proteins (Rajamanickam, V. et al., Chromatography, 2:195-212, 2015).

The monolithic matrix may be derived from a variety of materials, such as but not limited to, polymethacrylate, polyacrylamide, polystyrene, silica and cryogels. The monolithic matrix may be activated to promote coupling to a reactive moiety. Coupling to the activated monolithic matrix may occur, for example, through the formation of a covalent bond between the activated monolithic matrix and the reactive moiety. In some embodiments, the monolithic matrix is activated to couple to a primary amine group. Activation of the monolithic matrix can be accomplished through any appropriate methods known in the art (see, e.g., Pfaunmiller, E. et al., Anal. Bioanal. Chem., 405:2133-45, 2013; Hermanson, G., Bioconiugate Techniques, 3^{rd} Ed., 2013). Non-limiting examples of activation agents include carbonyldiimidazole (CDI), epoxy ethylenediamine (EDA), carbodiimide, aldehyde, anhydride, imidoester and NHS ester.

As used herein, the term “ligand” refers to a molecule that preferentially binds, covalently or non-covalently, to a molecule or material of interest. The ligands described herein can further comprise a reactive moiety capable of coupling to a monolithic matrix. An “oligo-dT ligand” is an oligo-dT probe. A “probe” refers to a ligand that selectively interacts, e.g., binds to or hybridizes with, a desired interaction partner, e.g., a specific polynucleotide sequence. A ligand can itself be a polynucleotide, e.g., an oligo-dT probe or an oligonucleotide, that, for example, specifically hybridizes to a sequence of interest, e.g., a poly-A tail or a sequence specific to the polynucleotide to be purified.

An oligo-dT probe consists of a chain of thymine bases or uracil bases or chemically modified bases of any length appropriate to specifically bind to the poly-A tail of mRNA. Non-limiting examples of oligo-dT probes include oligomers of the formula dT_{Y}, wherein Y is a whole number between 5 and 30. In specific embodiments, the oligo-dT probe is dT₁₅, dT₁₈, dT₂₀, dT₂₅ or dT₃₀.

The ligands described herein are coupled or attached to the solid support monolith matrix via a reactive moiety. The monolith can be activated, thereby allowing for coupling to the ligand via the active moiety of the ligand. In a particular embodiment, the monolithic matrix is activated with an activation agent to allow coupling to amine groups, and the reactive moiety of the ligand is a primary amine. In one embodiment the activation agent is carbonyldiimidazole.

In some embodiments, the ligand further comprises a carbon linker positioned between the reactive moiety and the oligo-dT probe. Selection of the length of the carbon linker is within capabilities of the skilled person. Non-limiting examples of carbon linkers include linkers of the formula C_{X}, wherein X is any whole number between 5 and 50. In specific embodiments, the carbon linker is C₆, C₇, C₈, C₉, C₁₀, C₁₁, C₁₂, C₁₃, C₁₄ or C₁₅.

In some embodiments, the ligand is NH₂—C_{X}-dT_{Y}, wherein C_{X} is a carbon chain of length X, and X is a whole number between 5 and 50; and dT_{Y} is an oligo-dT probe of length Y, and Y is a whole number between 1 and about 100, about 5 and about 50, about 10 and about 30, about 7 and about 26, about 18 and about 24, or between about 5 and about 25. In a particular embodiment the ligand is NH₂—C₁₂-dT₁₈.

Also described herein is a method of preparing a column for purifying mRNA from a sample, the method comprising treating a monolithic matrix with an activating agent to produce an activated monolithic matrix; and incubating the activated monolithic matrix in the presence of a ligand comprising a reactive moiety and a polynucleotide, e.g., an oligonucleotide, e.g., an oligo-dT probe. In some embodiments the ligand further comprises a carbon linker positioned between the reactive moiety and the polynucleotide probe. In some embodiments, the reactive moiety is a primary amine. In some embodiments, the activating agent is carbonyldiimidazole.

Also described herein are methods for purifying polynucleotides, e.g., oligonucleotides, e.g., mRNA or siRNA from a sample. Such methods include, for example, a) loading a sample onto a column comprising: i) a monolithic matrix with an attached ligand comprising: A) a reactive moiety coupled to the monolithic matrix, and B) a polynucleotide, e.g., oligo-dT, probe; b) washing the column; c) eluting the polynucleotide from the column; and d) collecting at least one elution fraction from the column. In one embodiment, step b) comprises washing the column with at least one wash buffer. In another embodiment, step c) comprises eluting the polynucleotides from the column with at least one elution buffer. In another embodiment, the elution fractions of step d) contain mRNA. In some embodiments, the wash buffer contains a salt concentration between about 150 mM to about 1 M. In particular embodiments, the wash buffer contains a salt concentration of at least about 200 mM, at least 400 mM or at least about 750 mM. In some embodiments, the elution buffer contains a salt concentration between 0 and about 100 mM. As used herein, the term “about” means plus or minus 10% of the numerical value of the number with which it is being used. In particular embodiments the elution buffer has a salt concentration of 100 mM or less. In particular embodiments the wash buffer comprises one or more salts selected from sodium sulfate, sodium chloride and sodium phosphate.

In one embodiment, the elution buffer is water. In another embodiment, the elution buffer comprises Tris. Tris buffer may be used at a concentration from about 1 mM to about 20 mM. In a particular embodiment, the elution buffer comprises 10 mM Tris.

Selection of the flow rate of the column is within capabilities of the skilled person. In some embodiments, the flow rate of the column is from about 1 mL/min to about 5 mL/min. In particular embodiments the flow rate is at least 2 mL/min, at least 3 mL/min or at least 4 mL/min.

The molecule or material of interest is separated from contaminants, and can come off the column in any of the flow-through, wash or elution fraction, depending on the nature of the molecule or material of interest and the major contaminant(s).

The following examples are included for illustrative purposes only and are not intended to limit the scope of the claims.

### EXAMPLES

### Example 1

Polynucleotides can be applied to monolith matrices as described herein for mRNA. The mRNA transcripts used in this example are described in Table 1; additional mRNA transcripts purified by the methods described herein are described in Table 2. Transcripts used were either LiCl precipitated or used straight after the transcription reaction following EDTA treatment.

TABLE 1
Characteristics of mRNA used in
purification studies.

| | Transcript | Poly-A |
|---|---|---|
| RNA ID | length | tail |
| RNA001 | 1837 | Yes |
| RNA021 | 452 | No |
| RNA023 | 971 | Yes |

CDI (carbonyldiimidazole or carboxydiimidazole)-activated monolith disk columns (0.34 mL) were purchased from BIA Separations through High Purity New England (Smithfield, R.I.). Ligands for immobilization on the CDI-monolithic columns were designed and purchased from Integrated DNA Technologies (Coralville, Iowa). Two ligands were used in these studies: an NH₂—C₁₂-dT₁₈ ligand, containing a primary amine followed by a 12-carbon linker chain and 18 deoxythymine bases; and an NH₂—C₆-dT₁₈ ligand, containing a primary amine followed by a 6-carbon linker chain and with 18 deoxythymine bases.

Experiments were run using a GE AKTA Avant 25 preparative chromatography system (GE Healthcare Life Sciences).

TABLE 2
Poly-A containing mRNA of various lengths
purified to >90% purity by oligo-dT

| | Transcript |
|---|---|
| ID | bases |
| RNA023 | 971 |
| RNA025 | 1909 |
| RNA027 | 1924 |
| RNA034 | 832 |
| RNA037 | 1432 |
| RNA181 | 1467 |
| RNA385 | 1642 |

### Oligo-dT Immobilization to a Monolithic Matrix

A syringe was used to load the oligo-dT ligand onto the monolithic column. All steps were performed at room temperature. The CDI disk column was assembled in the housing according to the manufacturer's instructions. The assembled column was flushed with at least 10 column volumes (CV) of Milli-Q water. The column was then equilibrated with at least 10 CV of suitable buffer (0.5 M Na Phosphate pH 8.0).

The oligo-dT ligand was dissolved in 0.5 M sodium phosphate (pH 8.0) to a final stock concentration of about 100 mg/mL. Then, 1.5-2.0 mL of ligand was diluted to 3 mg/mL with equilibration buffer and was pushed through the column using a syringe to completely fill the monolith channels. The column was then disconnected from the syringe and sealed with blind fittings. The column was stored at room temperature for 20-24 hours.

Following incubation with the oligo-dT ligand, the column was rinsed with at least 10 CV of suitable buffer (0.5 M Na Phosphate pH 8.0), and the column was then flushed with at least 10 CV Milli-Q water. The column was equilibrated with loading buffer (50 mM sodium phosphate, 750 mM sodium sulfate, 10 mM EDTA pH 7.0) for testing with samples of RNA.

### Purification Testing

Initial testing of mRNA binding to the monolithic column with immobilized oligo-dT ligand was done as described in Table 3, in the order stated.

TABLE 3
Initial purification process with oligo dT-immobilized monolithic column

| | | Flow rate | |
|---|---|---|---|
| Step | Buffer | ml/min | CV |
| Clean | 10 mM sodium hydroxide | 2 | 10 |
| Equilibrate | 750 mM sodium sulfate, 50 mM sodium | 2 | 10 |
| | phosphate, 10 mM EDTA pH 7.0 | | |
| Load | RNA | 1 | |
| Wash | 750 mM sodium sulfate, 50 mM sodium | 1 | 6 |
| | phosphate, 10 mM EDTA pH 7.0 | | |
| Wash 2 | 50 mM sodium phosphate, 10 mM | 1 | 25 |
| | EDTA pH 7.0 | | |
| Elution | Ultra-pure water | 1 | 15 |
| Clean | 10 mM sodium hydroxide | 1 | 15 |
| Clean | 10 mM sodium hydroxide | 2 | 10 |

The starting buffers used for purification testing were: Buffer A: 50 mM sodium phosphate, 1.0 M Na₂SO₄, 10 mM EDTA pH 7.0; and Buffer B: 50 mM sodium phosphate, 10 mM EDTA pH 7.0. Fractions from the flow-through were desalted as needed and analyzed appropriately.

### Example 2: Purification of mRNA Using Amino-Linked Oligo-dT Probe Immobilized on an Activated Monolithic Column

### Initial Binding Experiments

Initial conditions for testing purification of mRNA on the NH₂—C₁₂-dT₁₈ immobilized monolithic column were designed using a high salt binding buffer. The RNA bound to the monolith in 750 mM sodium sulfate, 50 mM phosphate buffer, 10 mM EDTA at pH 7.0 and was eluted with water. Various salt conditions were tested and are listed in Table 4. These experiments were completed using LiCl purified material.

TABLE 4
Initial binding results of RNA to C12-oligo d(T)18 immobilized monolith

| Sample | Load and Wash 1 Buffer Components | Binding Result |
|---|---|---|
| RNA001 | 750 mM sodium sulfate, 50 mM sodium | Bound and eluted |
| | phosphate, 10 mM EDTA, pH 7.0 | with water |
| RNA001 | 400 mM sodium sulfate, 50 mM sodium | Bound and eluted |
| | phosphate, 10 mM EDTA, pH 7.0 | with water |
| RNA001 | 200 mM sodium sulfate, 50 mM sodium | Bound and eluted |
| | phosphate, 10 mM EDTA, pH 7.0 | with water |

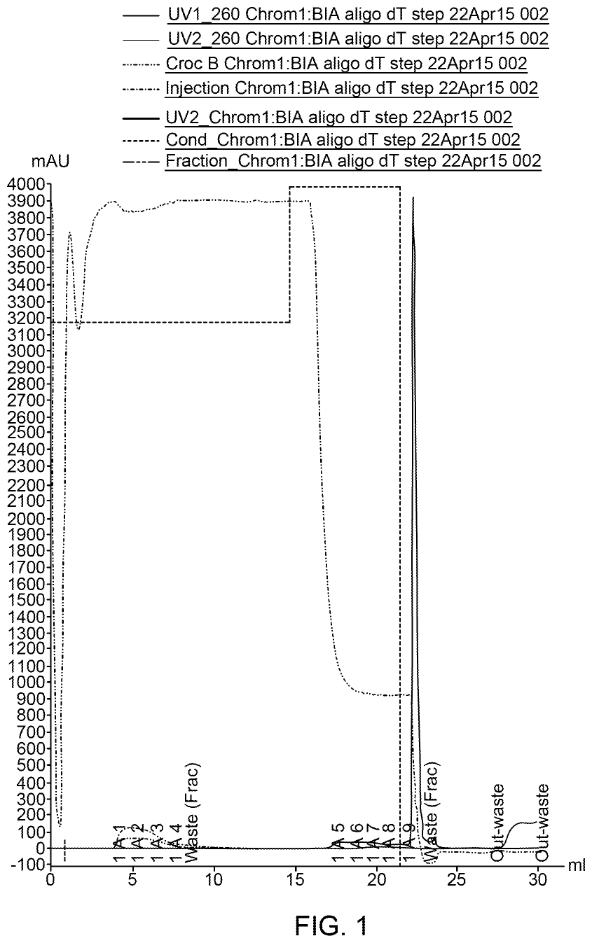

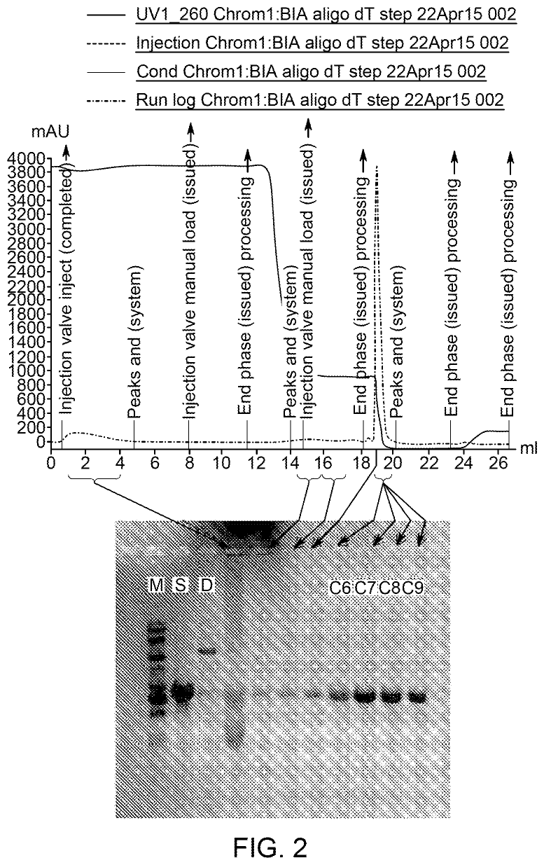

A chromatogram of LiCl precipitated RNA001 bound at 200 mM sodium sulfate buffer is shown in FIG. 1. A glyoxyl gel was used to visualize fractions from the RNA001 chromatography, and can be seen in FIG. 2. The flow through fractions (1A1-1A4) were combined and concentrated ten-fold for this gel. The majority of the flow through material was not full-length RNA. The gel indicates that 50 mM sodium phosphate, 10 mM EDTA pH 7.0 (no sodium sulfate) eluted off a small amount of product. Once the flow was shifted to ultra-pure water, the majority of RNA was eluted in a single peak of RNA, and was seen in fractions 1 C6 to 1C9 (labeled C6, C7, C8 and C9 in FIG. 2). Gel analysis of the chromatography fractions showed removal of impurities, specifically DNA template and abortives of the RNA that are missing the poly-A tail.

Following these initial experiments, RNA021 (which had no poly-A tail to interact with the oligo-dT ligand) was assessed using the immobilized monolith disk column. RNA021 was tested using the conditions described in Table 5, in the order stated, and compared to the RNA001 that contains a poly-A tail.

TABLE 5
Process conditions fortesting of RNA021 on the C12 oligo dT(18) monolith

| | | Flow rate | |
|---|---|---|---|
| Step | Buffer | ml/min | CV |
| Clean | 10 mM sodium hydroxide | 2 | 10 |
| Equilibrate | 200 mM sodium sulfate, 50 mM sodium | 2 | 10 |
| | phosphate, 10 mM EDTA pH 7.0 | | |
| Load | RNA | 1 | |
| Wash | 200 mM sodium sulfate, 50 mM sodium | 1 | 6 |
| | phosphate, 10 mM EDTA pH 7.0 | | |
| Elute | 50 mM sodium phosphate, 10 mM | 1 | 25 |
| | EDTA pH 7.0 | | |
| Water flush | Ultra-pure water | 1 | 15 |
| Clean | 10 mM sodium hydroxide | 1 | 15 |

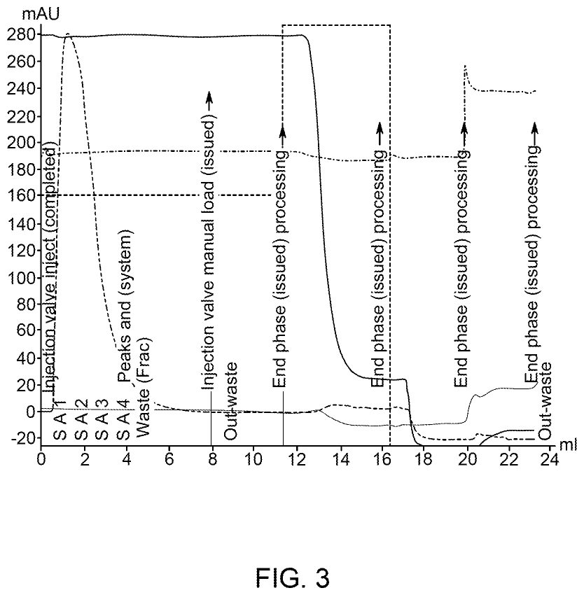

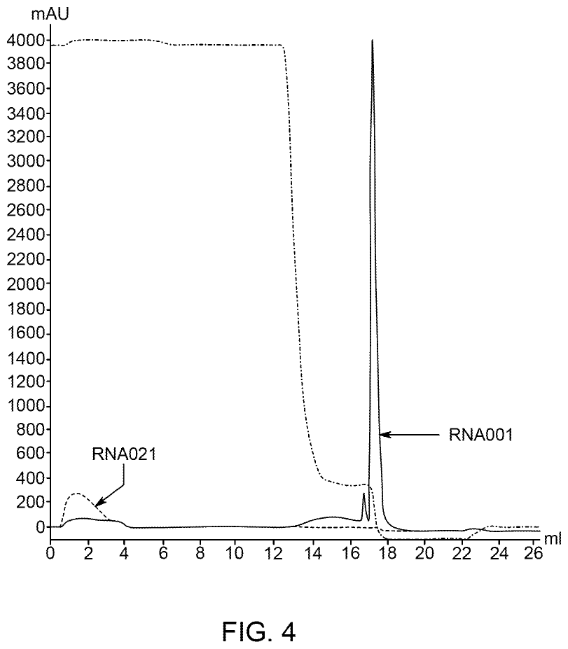

The resulting chromatogram is shown in FIG. 3. RNA elution would be expected to start at approximately 17 mL FIG. 4 shows an overlay of the RNA021 (without poly-A tail) and RNA001 (with poly-A tail) chromatograms run using the same conditions. RNA elution would be expected to start at approximately 17 mL

### Example 3: Purification of Transcription Reactions

The NH₂—C₁₂-dT₁₈ immobilized monolithic column was evaluated using transcription reactions that had not been purified further following in vitro transcription. Chromatography conditions for these samples are described in Table 6. The RNA loads in Table 6 were transcription reactions treated with EDTA only. There were slight adjustments made to the CV amount for the wash (increased from 6 to 10 CV) and elution (decreased from 25 to 15 CV).

TABLE 6
Chromatography process for RNA transcription reactions.

| | | Flow rate | |
|---|---|---|---|
| Step | Buffer | ml/min | CV |
| Clean | 10 mM sodium hydroxide | 2 | 10 |
| Equilibrate | 200 mM sodium sulfate, 50 mM sodium | 2 | 10 |
| | phosphate, 10 mM EDTA pH 7.0 | | |
| Load | RNA | 1 | 8 ml |
| Wash | 200 mM sodium sulfate, 50 mM sodium | 1 | 10 |
| | phosphate, 10 mM EDTA pH 7.0 | | |
| Elute | 50 mM sodium phosphate, 10 mM | 1 | 15 |
| | EDTA pH 7.0 | | |
| Water flush | Ultra-pure water | 1 | 15 |

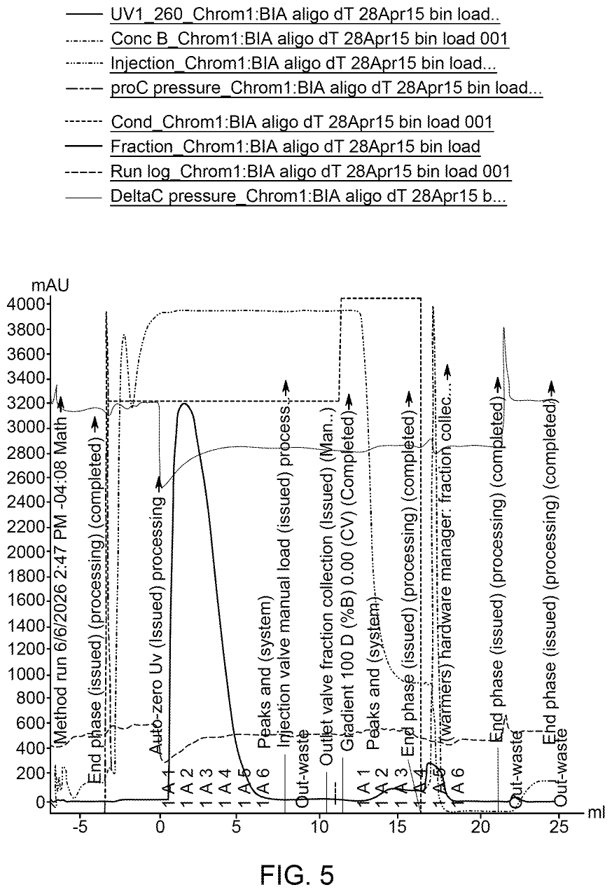

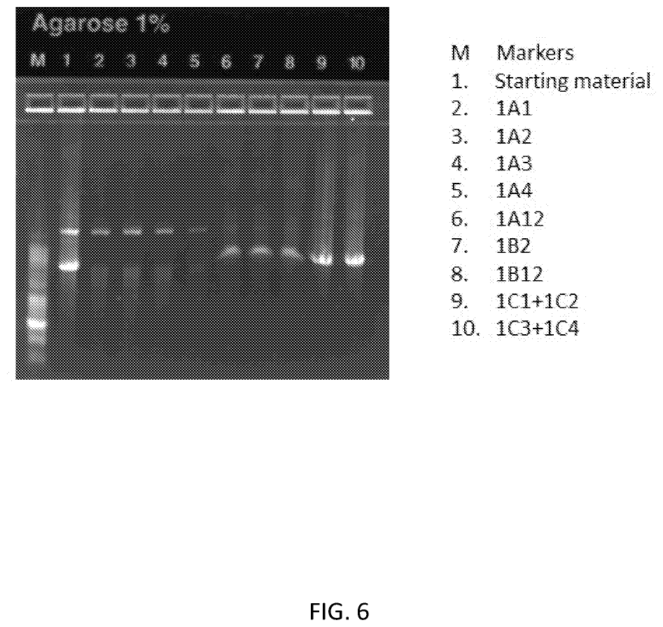

The resulting chromatogram (FIG. 5) shows a large flow-through (FT) peak from the remaining reaction components and products such as excess NTPs that do not bind the column. FT fractions were desalted and run over a 1% E-GEL™ (precast agarose gel) along with the peak fractions. FIG. 6 shows the results analyzed by agarose gel. The FT fractions (labeled 1 A1 through 1 B12 in FIG. 6) contain linearized DNA and what appear to be abortive RNA sequences. The peak fractions (labeled 1 C1 through 1 C4 in FIG. 6) showed a lower-running diffuse band, which disappears following treatment with RNase A, indicating that it is RNA.

The results indicate that a transcription reaction can be applied directly to an immobilized monolithic column and purified to the same degree as applying RNA that has been initially purified by LiCl precipitation and buffer exchange.

### Example 4: Increased Flow Rates

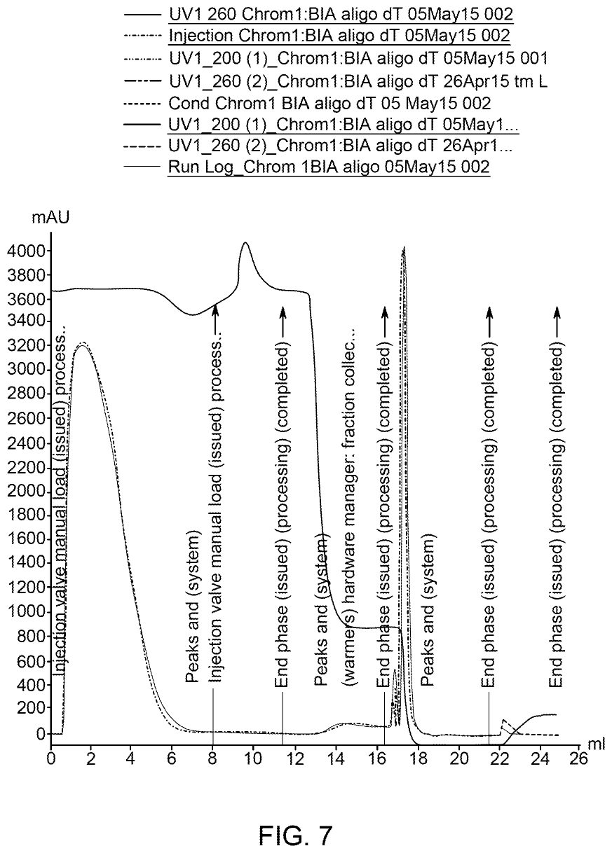

To test the influence of flow rate on mRNA purification over the ligand-immobilized monolithic column, flow rates up to 4 mL/minute were tested using the same samples and process conditions. Pressures were below acceptable levels for all flow rates. Overlays of chromatograms at 2, 3 and 4 mL/minute (FIG. 7) showed only minor differences between runs, indicating that increases in flow rate did not change the elution profile of the run. Exemplary column scales and operating parameters utilizing oligo-dT immobilization are described in Table 7.

TABLE 7
Monolith column sizes and operating parameters

| Column | | | | | |
|---|---|---|---|---|---|
| volume | Recommended | | Max | | Max |
| (mL) | flow rates | | mL/min | | CV/min |
| 0.34* | 2-4 | mL/min | 6 | mL/min | 18 |
| 1* | 1-5 | mL/min | 16 | mL/min | 16 |
| 8* | 8-60 | mL/min | 100 | mL/min | 12.5 |
| 80* | 80-240 | mL/min | 400 | mL/min | 5 |
| 800 | 200-1300 | mL/min | 2000 | mL/min | 2.5 |
| 8000 | 2000-10000 | mL/min | 10000 | mL/min | 1.25 |

*denotes columns have been tested.

### Example 5: Testing RNA with and without a Poly-A Tail

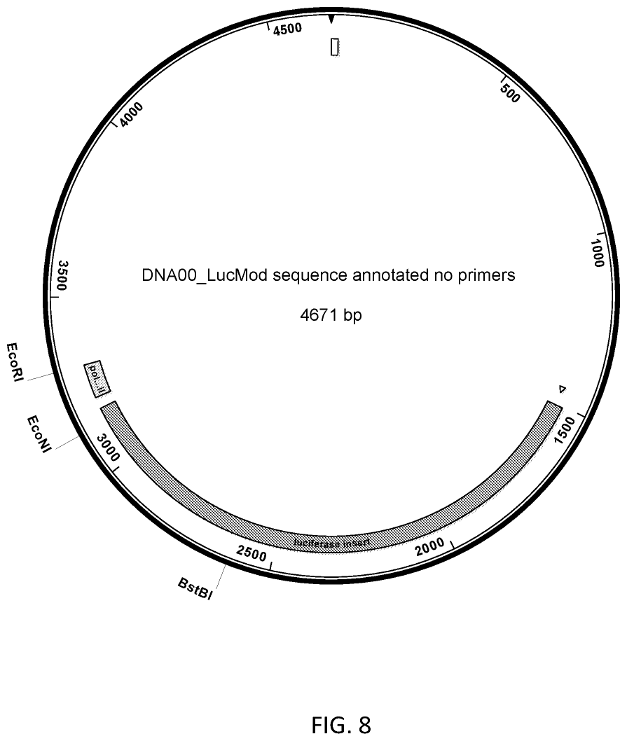

Binding of RNA transcripts where the poly-A tail was absent from the RNA001 transcript was accomplished by digesting the DNA template (DNA001, FIG. 8) for RNA001 using restriction enzymes that cut upstream of the sequence coding for the poly-A tail.

Table 8 describes the resulting RNA transcripts following digestion and transcription of DNA001.

TABLE 8
Resulting transcripts from digested DNA001

| | RNA | |
|---|---|---|
| Restriction | product | Poly-A |
| Enzyme | length | tail |
| EcoR1 | 1837 | Yes |
| EcoN1 | 1662 | No |
| BstB1 | 1150 | No |

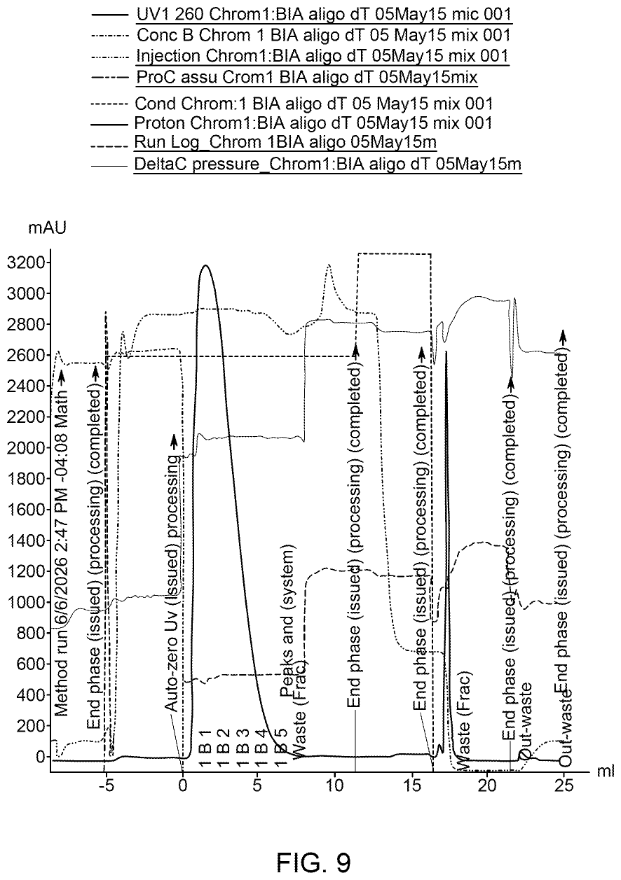

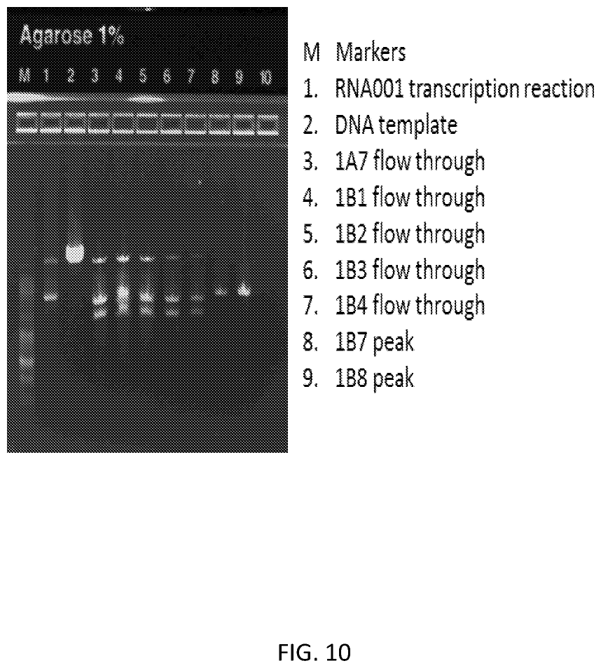

Following transcription of these templates, the single RNA was loaded on the ligand-immobilized monolithic column using the conditions listed in Table 5. RNA transcripts without poly-A tails flowed through when applied to the column. An equal parts mixture of the three RNA transcripts listed in Table 8 were applied to the monolithic column. The resulting chromatogram is shown in FIG. 9. Peak fractions collected were applied to a 1% E-GEL™ (precast agarose gel) to visualize the FT components and elution peak (FIG. 10).

The shorter transcripts lacking a poly-A tail do not bind to the column and are found in FT fractions (FIG. 10, lanes 3-7). DNA is also observed in the FT (FIG. 10, lanes 3-7). Only the full-length RNA that has the poly-A tail is observed in the elution peak (FIG. 10, lanes 8 and 9). The results confirm that the poly-A tail is required for the bind/elute purification observed in EXAMPLE 2.

### Example 6: Alternate Linkers

To evaluate the effect of the ligand linker on purification efficiency of RNA containing a poly-A tail, a shorter linker (C₆ vs C₁₂) between the amino group and oligo-dT probe was tested. The ligand was attached to a new monolithic column using the same method as described in EXAMPLE 1. Once the newly immobilized ligand was attached, the column was washed and tested with RNA001 to compare binding to the C₁₂ linker version of the ligand.

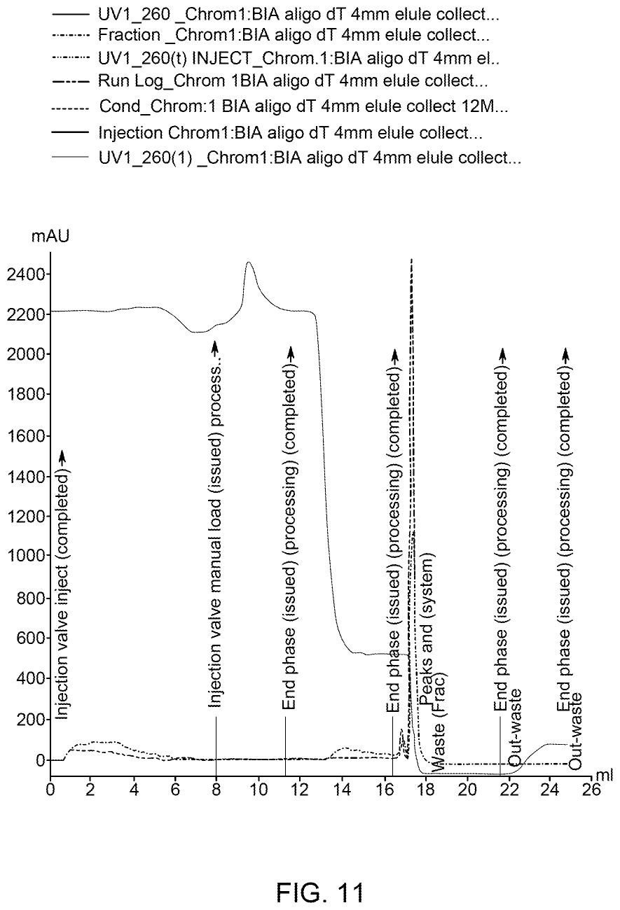

FIG. 11 shows an overlay of chromatograms of the same RNA run with the two different linkers. For the C₆-linker column, there was higher A₂₆₀ absorbance observed in the FT and the low salt wash as compared to the material run over the C₁₂-linker column. In addition, the C₁₂-linker purification resulted in about 100% yield (peak labeled * in FIG. 11) vs. the C₆ linker, which was about 65% yield (peak labeled ** in FIG. 11). The difference may be due to the shorter linker arm and proximity of the dT being closer to the monolith hindering full complementary binding of the poly-A tail to the dT stretch of nucleotides.

### Example 7: Salt Comparison

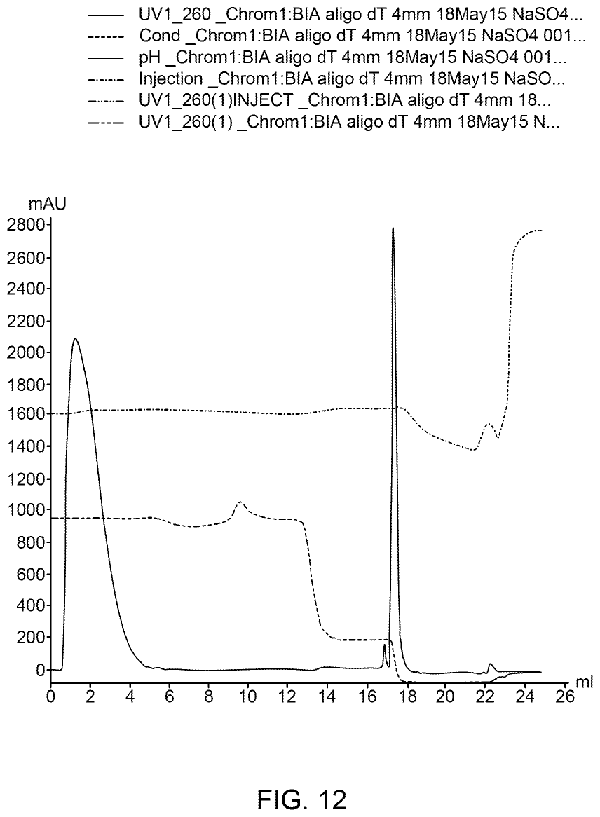

To evaluate the effect of different salts on the purification efficiency, sodium sulfate was replaced with sodium chloride in the equilibration/loading and wash buffers, keeping the phosphate buffer, EDTA and pH the same. FIG. 12 shows an overlay of traces from each of the two salts (* indicates the overlapping traces in FIG. 12). With the same process conditions, the chromatograms rendered from the two salts tested showed no difference in purification of the RNA.

### Example 8: Elution Conditions

Experiments for the binding and elution conditions for the RNA from oligo-dT monolithic columns looked at loading and washing in a high salt (at least 200 mM) followed by removal of the salt component of the buffer system. The remaining phosphate and EDTA did not elute the RNA; however the subsequent ultra-pure water flush eluted the RNA in a single tight peak. The absence of conductivity proved to be a potent elution condition.

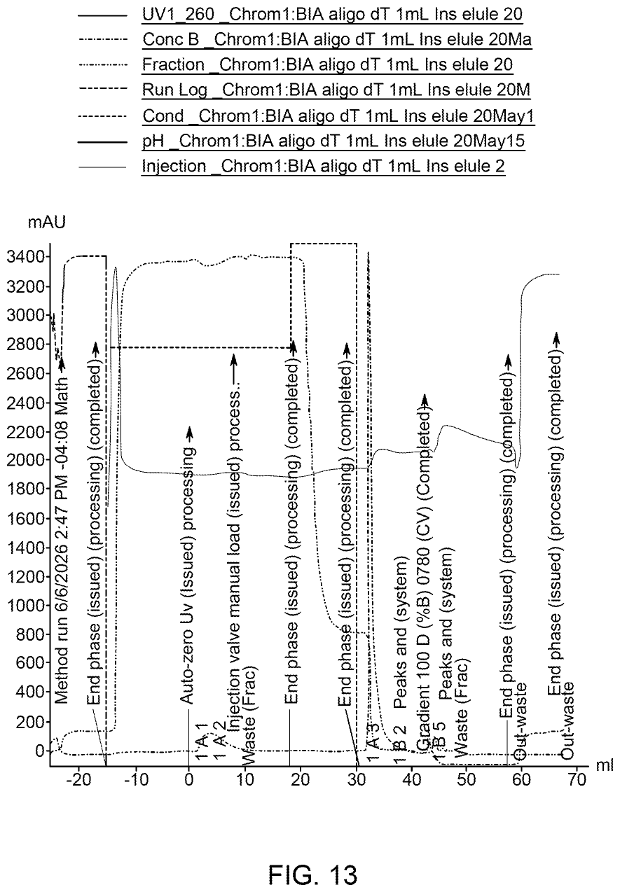

Analysis of the chromatograms indicated that the pH of the elution drifted upwards from pH 7 to as high as pH 9. To control the pH during the elution step, a low conductivity buffer (10 mM Tris pH 7.5) was tested on the 1 mL NH₂—C₁₂-oligo-dT₁₈ monolith and implemented for elution of the RNA. FIG. 13 shows a chromatogram trace of the elution step with Tris buffer. The results indicated that mRNA can be eluted from the ligand-immobilized monolithic column using either water or Tris buffer.

### Example 9

Although mRNA has a short half-life in vivo, high doses of free mRNA can transfect cells and tissues. Additionally, unwanted systemic introduction of mRNA can trigger an immune response before degradation and clearance.

Lipid Nanoparticles (LNPs) can be used to encapsulate and deliver, for example, mRNA. LNPs typically have at least 80% encapsulation of mRNA, i.e., mRNA that is located inside of an LNP as opposed to outside (“free” mRNA). This EXAMPLE evaluates the ability of monolith, oligo-dT purification to remove unencapsulated mRNA to produce a purified LNP.

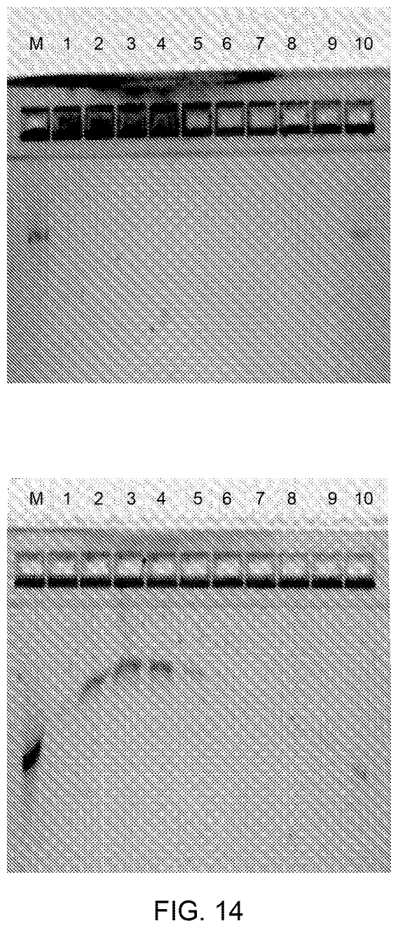

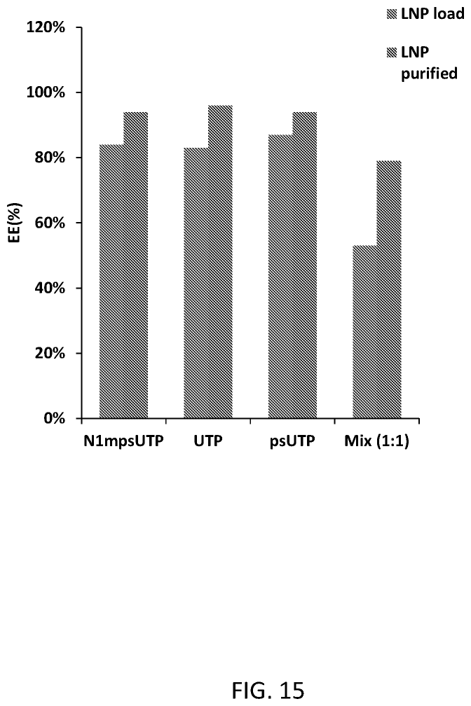

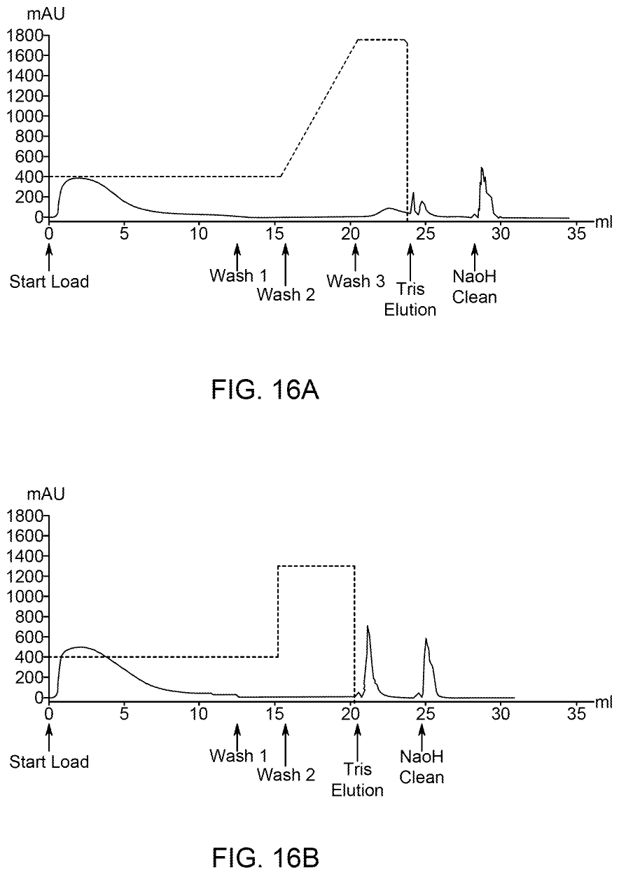

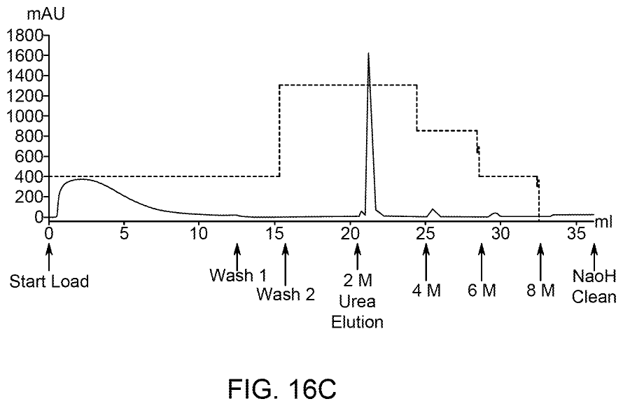

The results (FIGS. 14 and 15) demonstrate the general ability of oligo-dT based chromatography to separate free mRNA (with and without chemical modifications) from mRNA formulated in LNPs. This work could extend to purification of LNPs from other impurities besides mRNA if different affinity or immobile phase conditions are used. These data also indicate the strategy for purifying formulated polynucleotides extends to any polynucleotide, e.g., mRNA delivery system, not just LNPs.

LNPs were formulated with mRNA with encapsulation efficiency greater than 80%. Chromatograms and gels demonstrated that LNPs eluted in the flow-through fractions (FIG. 14). Free mRNA bound to the oligo dT column and eluted with salt adjustment to the mobile phase. Assessment of LNPs before and after purification showed no impact on size and polydispersity (Table 9). LNP yield was −70% after purification (FIG. 15)

TABLE 9
LNP characterization before and after purification.

| | LNP Load | | LNP purified | |
|---|---|---|---|---|
| | Diameter | | Diameter | |
| Sample | (nm) | PDI* | (nm) | PDI |
| N1- | 92 | 0.04 | 96 | 0.05 |
| methylpseudouridine | | | | |
| Uridine | 95 | 0.08 | 89 | 0.03 |
| Psuedouridine | 93 | 0.05 | 95 | 0.07 |

### OTHER EMBODIMENTS

It is to be understood that the foregoing description is intended to illustrate and not limit the scope of the invention, which is defined by the scope of the appended claims. Other aspects, advantages, and modifications are within the scope of the following claims. References cited herein are herein incorporated by reference in their entireties.

1. A method of purifying a polynucleotide from a sample, the method comprising:
a) loading the sample onto a monolithic matrix comprising a ligand comprising:
i) a reactive moiety coupled to the monolithic matrix, and
ii) an affinity moiety having the formula —C₁₂-dT₁₈, wherein dT₁₈ is a poly-A binding oligomer consisting of 18 deoxythymidine monomers that binds to the polynucleotide, —C₁₂— is a 12-carbon linker chain positioned between the poly-A binding oligomer and the reactive moiety, wherein the ligand is immobilized to the monolithic matrix via the reactive moiety;
b) allowing for the polynucleotide to bind to the ligand; and
c) eluting the polynucleotide from the monolithic matrix after one or more contaminants have been substantially separated from the bound polynucleotide.

2. The method of claim 1, wherein the reactive moiety is a primary amine.

3. The method of claim 1, wherein reactive moiety is capable of undergoing a coupling reaction with a monolithic matrix that has been activated with an activating agent selected from carbonyldiimidazole, epoxy, ethylendiamine, carbodiimide, aldehyde, anhydride, imidoester and NHS ester.

4.-5. (canceled)

6. The method of claim 1, wherein the polynucleotide is mRNA.

7. The method of claim 6, wherein the affinity moiety is a poly-A binding probe.

8. (canceled)

9. The method of claim 1, wherein the ligand has the formula NH₂—C₁₂-dT₁₈ prior to undergoing a coupling reaction with a monolith matrix, wherein —C₁₂— is a 12-carbon linker chain.

10.-15. (canceled)

16. A method of separating a formulated polynucleotide from free polynucleotide, the method comprising:
a) loading a sample onto a monolithic matrix comprising a ligand comprising:
i) a reactive moiety coupled to the monolithic matrix, and
ii) an affinity moiety having the formula —C₁₂-dT₁₈, wherein dT₁₈ is a poly-A binding oligomer consisting of 18 deoxythymidine monomers that binds to the free polynucleotide but not the formulated polynucleotide, —C₁₂— is a 12-carbon linker chain positioned between the poly-A binding oligomer and the reactive moiety, wherein the ligand is immobilized to the monolithic matrix via the reactive moiety; and
b) collecting the formulated polynucleotide from the column while the free polynucleotide remains immobilized on the monolithic matrix.

17. The method of claim 16, wherein the monolithic matrix is contained in a column.

18. The method of claim 16, wherein the formulated polynucleotide is a formulated mRNA.

19. The method of claim 18, wherein the mRNA is formulated in a lipid nanoparticle.

20. The method of claim 18, wherein the mRNA comprises a poly-A tail.

21. (canceled)

22. The method of claim 20, wherein the ligand has the formula NH₂—C₁₂-dT₁₈ prior to undergoing a coupling reaction with a monolith matrix, wherein —C₁₂— is a 12-carbon linker chain.

23.-24. (canceled)

25. The method of claim 16, further comprising eluting the free polynucleotide from the monolithic matrix via a liquid phase by reducing the ionic strength of the liquid phase.

26. A preparative-scale column for purifying a polynucleotide from a sample, said column comprising:
c) a monolithic matrix; and
d) a ligand coupled to the monolithic matrix, wherein the ligand comprises a reactive moiety and an affinity moiety having the formula —C₁₂-dT₁₈, wherein —C₁₂— is a 12-carbon linker chain positioned between the reactive moiety and dT₁₈, wherein the dT₁₈ is a poly-A binding oligomer consisting of 18 deoxythymidine monomers, and wherein the affinity moiety is coupled to the monolithic matrix at the carbon linker via the reactive moiety;
wherein the column has a column volume of at least 0.34 mL and is configured to allow a flow rate of at least 1 mL/min.

27. The preparative-scale column of claim 26, wherein the reactive moiety is a primary amine.

28. The preparative-scale column of claim 26, wherein the reactive moiety is capable of undergoing a coupling reaction with the monolithic matrix that has been activated with an activating agent selected from carbonyldiimidazole, epoxy, ethylendiamine, carbodiimide, aldehyde, anhydride, imidoester and NHS ester.

29.-32. (canceled)

33. The preparative-scale column of claim 26, wherein the ligand has the formula NH₂—C₁₂-dT₁₈ prior to undergoing a coupling reaction with a monolith matrix, wherein —C₁₂— is a 12-carbon linker.

34.-35. (canceled)

36. The preparative-scale column of claim 26, wherein the column volume is at least 0.34 mL and the column is configured to allow a flow rate of at least 2 mL/min.

37. The preparative-scale column of claim 26, wherein the column volume is at least 1.0 mL and the column is configured to allow a flow rate of at least 1 mL/min.

38. The preparative-scale column of claim 26, wherein the column volume is at least 8.0 mL and the column is configured to allow a flow rate of at least 8 mL/min.

39. The preparative-scale column of claim 26, wherein the column volume is at least 80.0 mL and the column is configured to allow a flow rate of at least 80 mL/min.

40. The preparative-scale column of claim 26, wherein the column volume is at least 800.0 mL and the column is configured to allow a flow rate of at least 200 mL/min.

41. The preparative-scale column of claim 26, wherein the column volume is at least 8000.0 mL and the column is configured to allow a flow rate of at least 2000 mL/min.

42. The preparative-scale column of claim 26, wherein the column volume is from 0.34 mL to 8000 mL.

43. The preparative-scale column of claim 26, wherein the column is configured to allow a flow rate of 1 mL/min to 10,000 mL/min.
